(19) United States
(12) Patent Application Publication
(10) Pub. No.: US 2002/0035131 A1
(43) Pub. Date: Mar. 21, 2002

(54) Regimen and kit for amelioration of premature ejaculation

(75) Inventors: Ragab El-Rashidy, Deerfield, IL (US); Chris McMahon, Sydney (AU)

Correspondence Address: OLSON & HIERL, LTD., 36th Floor, 20 North Wacker Drive, Chicago, IL, 60606, US

(73) Assignee: Pentech Pharmaceuticals, Inc.

(21) Appl. No.: 09/970,293

(22) Filed: Oct. 3, 2001

Related U.S. Application Data
(62) Division of application No. 09/306,273, filed on May 6, 1999, now abandoned.

(51) Int. Cl. A61K 31/4525

(52) U.S. Cl. 514/321

(57) ABSTRACT

Male patients suffering from premature ejaculation dysfunction are treated by an oral therapy regimen of administration of paroxetine substantially within several hours before sexual intercourse. The beneficial effect of the paroxetine treatment can be optimized and maintained by a combination oral therapy regimen in which the patient converts to continual maintenance paroxetine administration after an initial loading period of daily doses of paroxetine taken over of relatively short duration.

### FIELD OF THE INVENTION

This invention relates to the treatment of a sexual dysfunction and, more particularly to a regimen and kit for ameliorating premature ejaculation in a human male patient.

### BACKGROUND OF THE INVENTION

Premature ejaculation (PE) is the most common male sexual disorder, affecting perhaps as many as 75% of men at some stage in their sexual lives. The Diagnostic and Statistical Manual of Psychiatry (DSM-IV) defines premature ejaculation as “persistent or recurrent ejaculation with minimal sexual stimulation before, on or shortly after penetration and before the person wishes it . . . ” which is associated with “. . . marked distress or interpersonal difficulty . . . ”. This disorder is usually primary and when secondary is often associated with a significant degree of marital turmoil.

Primary premature ejaculation is invariably psychogenic due to performance anxiety, fear or psychological trauma and current research suggests that some premature ejaculation may have a biogenic basis. Premature ejaculation has historically been treated predominantly by psychosexual counseling, which also requires the cooperation of the man's sexual partner. However, many men decline or fail to complete a trial of psychosexual counseling for a variety of personal reasons, such as fear of social stigma or unwillingness to attend counseling sessions, and the like. Other men may demand a quicker response than psychosexual counseling is reported to offer or do not have a sexual partner willing to attend the counseling sessions.

The selective serotonin reuptake inhibitor (SSRI) antidepressants, such as chlomipramine, fluoxetine, sertraline and paroxetine have recently emerged as alternative new treatments for patients with premature ejaculation. On the other hand, anti-anxiety drugs such as chlordiazepoxide (LIBRIUM®) and diazepam (VALIUM®) are not suitable for the treatment of premature ejaculation.

Delayed ejaculation is a common adverse effect of many psychotropic and antidepressant drugs which act centrally and/or locally to retard the psychoneurological control of ejaculation and subsequent orgasm. In general, antidepressants influence more than one neurotransmitter system and have affinity for multiple receptors. Animal studies have shown that the central neutrotransmitter serotonin has an inhibitory effect on sexual function, while dopamine is generally stimulatory. Sexual effects can occur through any shift in this serotonin-dopamine balance by an increase or decrease in either or both neurotransmitter.

This heterogeneity of action by SSRI antidepressants produces mixed effects, including those on the sexual response cycle. Sexual dysfunction associated with antidepressants, such as delayed and completely abolished ejaculation (anejaculation), has been a subject of numerous case reports, studies, and review articles. Because of the lack of abuse potential, relatively benign side effect profile, and fairly consistent reports of delayed ejaculation, SSRI antidepressants taken on a daily basis seem to offer a safe treatment option for patients with premature ejaculation, especially in cases of failed psychological treatment.

For example, the use of the SSRI antidepressant fluoxetine hydrochloride (PROZAC®) on a daily basis in this regard has been described in U.S. Pat. No. 5,151,448 to Crenshaw et al. A similar treatment, at a relatively lower daily dosage of active ingredient, has been described in U.S. Pat. No. 5,276,042 to Crenshaw et al. for the SSRI antidepressant paroxetine hydrochloride (PAXIL®). Other researchers have also reported significant improvement in ejaculatory control with daily (chronic) use of paroxetine. Waldinger, M. D., et al., in “Paroxetine treatment of premature ejaculation: a double-blind randomized, placebo-controlled study,” American Journal of Psychiatry, 151, 1377 (1994); and in “Ejaculation-retarding properties of paroxetine in men with primary premature ejaculation: a double-blind, randomized, dose-response study,” British Journal of Urology, 79(4); 592-5 (1997 April) reported studies with 17 men. See also, Ludovico, G. M., et al., “Paroxetine in the treatment of premature ejaculation,” British Journal of Urology, 77(6); 881-2 (1996 June) and McMahon C. G., “Treatment of premature ejaculation with paroxetine hydrochloride,” International Journal of Impotence Research, 9 (Supp. 1) S38:Abst. 36 (1999).

The use of paroxetine, administered orally in maintenance dosage amounts of about 20 to about 60 milligrams, on a chronic daily basis for as long as a male remains sexually active has been described by Crenshaw et al., in U.S. No. 5,276,042 as a treatment for premature ejaculation. There is a need and desire for an oral therapy regimen which avoids or minimizes the adverse effects and inconvenience associated with chronic daily dosing of paroxetine, however.

It has now been found, however, that ejaculatory control can be achieved, optimized, and continually maintained at relatively lower dosage levels of paroxetine in an oral therapy regimen.

### SUMMARY OF THE INVENTION

An oral therapy regimen employing paroxetine or a pharmaceutically acceptable acid addition salt thereof, administered ad lib within several hours, prior to sexual intercourse can delay the onset of ejaculation in a human male patient suffering from premature ejaculation dysfunction. The delay in onset of ejaculation in certain patients can be further optimized and maintained by a combination oral therapy regimen in which the male patient converts to paroxetine continual maintenance administration after an initial starter daily dosage of paroxetine taken over an initial loading period of relatively short duration, preferably followed by a paroxetine optimization period of about two to not more than about four weeks duration.

Also provided is a kit package containing discrete dosage forms of paroxetine or pharmaceutically acceptable salt thereof for use in practicing the oral therapy regimen in the home. The package preferably comprises an articulated strip of calendar cards arranged for and containing a designated regimen of dosages. A preferred package includes a paroxetine starter dosage strip, a paroxetine optimization dosage strip and a paroxetine continual maintenance dosage strip arranged in the foregoing sequential order.

The paroxetine starter dosage strip can contain seven paroxetine-containing dosage forms arranged in spaced apart relationship to one another on the strip to be taken daily orally on days 1-7 of the initial loading period. The paroxetine starter dosage strip can bear dosage day indicia useful for correlating a particular dosage to a particular day on a one to one starter dosage form relationship.

The paroxetine optimization dosage strip can contain at least 14 and up to and including 28 paroxetine-containing dosage forms arranged in spaced apart relationship to one another on the strip to be taken orally. The optimization period can be two to four weeks. Thus, the optimization dosage forms can be taken daily orally starting on day 8 through day 28, depending on the duration of the optimization period. The paroxetine optimization dosage strip can bear dosage day indicia useful for correlating a particular dosage to a particular day on a one to one optimization dosage form relationship. The amount of optimization dosage per optimization dosage form preferably is twice the amount of the starter dosage.

The paroxetine continual maintenance dosage strip can contain a plurality of paroxetine-containing dosage forms arranged in spaced apart relationship to one another on the strip, the amount of paroxetine dosage in at least every other dosage form being the same as the starter dosage. After the optimization period, a single continual maintenance dosage form can be taken orally ad lib within a period of about two to about 20 hours prior to engaging in sexual intercourse. Optionally, instructional indicia useful for correlating a particular dosage to a particular time for taking each dosage form to delay the onset of ejaculation by the patient during subsequent intercourse can be provided on the maintenance dosage strip. Preferred dosage forms are tablets and capsules.

In a preferred method aspect, paroxetine can be administered to the patient according to the following oral therapy regimen:

(A) first a starter dosage of a single dosage form containing paroxetine is taken by the patient daily over an initial loading time period of about seven days;

(B) next, an optimization dosage of a single dosage form containing twice the amount of paroxetine as the starter dosage is taken by the patient daily over an optimization time period of about two to about four weeks, preferably about three weeks; and then

(C) a continual maintenance dosage of a single dosage form containing the same amount of paroxetine as the starter dosage is taken by the patient within a period of about two hours to about 20 hours, preferably within about three to about six hours, prior to engaging in sexual intercourse.

A starter dosage and continual maintenance dosage amount can be about 5 to about 40 milligrams, preferably about 10 to about 30 milligrams paroxetine. A preferred optimization dosage amount can be about 10 to about 30 milligrams paroxetine.

Optionally, if the paroxetine starter dosage period is sufficient to delay the onset of premature ejaculation from that before treatment, the patient can omit the paroxetine optimization dosage period and convert to continual maintenance paroxetine dosage preferably taken at least every other day. Alternatively, a patient can practice an oral therapy of continual maintenance administration by taking a single dose of paroxetine of about 5 to about 40 milligrams within a period of about two to about 20 hours, preferably within about three to about six hours, prior to engaging in sexual intercourse.

The ejaculatory latency time (ELT) in normally potent male patients suffering from premature ejaculation dysfunction was beneficially prolonged over pre-treatment ejaculation time after treatment in a therapy regimen solely with continual maintenance dosage or after converting to continual maintenance.

Advantageously, the treatment regimen of this invention avoids the need for chronic daily dosing of paroxetine and minimizes the side effects associated therewith.

### BRIEF DESCRIPTION OF THE DRAWINGS

In the drawings,

FIG. 1 is a graphic illustration of a preferred dose regimen therapy scheme comprising a starter initial loading dosage period, an optimization dosage period and a continual maintenance dosage period;

FIG. 2 is a plan view of a kit embodiment for practicing the dose regimen shown in FIG. 1 showing an articulated strip of blister-packaged dosage forms;

FIG. 3 is a partial plan view of the continual maintenance dosage strip in the kit embodiment of FIG. 2 in which every dosage form contains an active amount of dosage; and

FIG. 4 is an alternate partial plan view of the continual maintenance dosage strip in the kit embodiment of FIG. 2 in which every other dosage form contains an active amount of dosage.

### DETAILED DESCRIPTION OF PREFERRED EMBODIMENTS

Paroxetine can be represented by the structural formula (A) shown below:

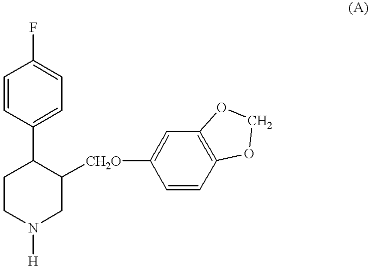

and is also known as trans-(−)-3-(1,3-benzodioxol-5-yloxy)methyl-4-(4-fluorophenyl) piperidine. The term “paroxetine” as used herein includes the free base form of this compound as well as the pharmaceutically acceptable acid addition salts thereof. Paroxetine hydrochloride is preferred and is commercially available under various trade designations, such as, PAXIL® from SmithKline Beecham and AROPAX® from SmithKline Beecham International Pharmaceuticals. PAXIL® tablets are commercially available in dosages of 10, 20, 30 and 40 milligrams paroxetine hydrochloride.

Useful dosage forms can be substantially solid articles, such as tablets, capsules and the like. Thus, other dosages, such as 6, 9, 12, 20, and 25 milligrams and the like, of paroxetine hydrochloride can be used in capsule form, for example.

Paroxetine reportedly has a relatively long half life of 24 hours allowing once daily dosing. Peak plasma levels are usually achieved within about 2-8 hours with steady state systemic levels occurring after about 7-14 days. Paroxetine undergoes extensive first pass metabolism principally to conjugates with glucuronic acid and sulphate which have no more than 1/50 the potency of their parent compound at inhibiting serotonin uptake. The only reported contraindication to use of paroxetine, apart from known hypersensitivity, is the concurrent use of monoamine oxidase inhibitors. Paroxetine selectively inhibits serotonin (5-HT) uptake in brain neurons, but unlike sertraline has little affinity for dopamine receptors and central β-adrenergic receptors. The most common side effects reported are sexual and gastrointestinal but a very occasional patient will experience the agitation and tremor seen with fluoxetine. Drug interactions with warfarin, tryptophan, dilantin have been reported.

The term “premature ejaculation” as used herein refers to ejaculation that occurs within 60 seconds of vaginal intromission. The efficacy of paroxetine in the method of this invention in delaying the onset of ejaculation was assessed in prospective studies with normally potent male patients suffering from premature ejaculation (“PE”). The patients were selected on the basis of a having baseline pretreatment ejaculation latency time (“ELT”) of less than about two minutes. An ELT of less than one minute was regarded as severe premature ejaculation dysfunction. In contrast, the average ELT of a normally potent male is about three to about four minutes. The baseline ELT of the patient was determined as the mean of measurements at a minimum of two acts of sexual intercourse prior to commencing treatment using a stopwatch operated by the patient.

Patients with erectile dysfunction, reduced sexual desire, inhibited male orgasm, chronic psychiatric or physical illness, alcohol or substance abuse and the use of medication, including psychotropic and anti-depression medication were excluded from trials. The patients were asked not to use condoms, topical penile anaesthetic creams or sprays. None of the men received any formal psychosexual counselling.

The term “continual maintenance” with respect to the inventive therapeutic method means that the male patient orally ingested a single dose of paroxetine ad lib within a period of about two to about 20 hours, and more preferably within a period of about three to about six hours, prior to sexual intercourse. The term “acute” with respect to the dosing means that the patient ingested an oral dose of paroxetine daily after breakfast or lunch for a period of at least one week. The patients were supplied with an ejaculation diary and were asked to record their frequency of coitus, quality of erection and orgasm, and to record their ELT using a stopwatch. The patients were required to attempt coitus on at least two occasions each week at home. In some trials, the patient's partner was also instructed on the use of the stopwatch and asked to record her recollection of the timing of the male patient's ejaculation.

For continual maintenance therapy the patient can employ a dose of paroxetine taken in an amount ranging about 5 to about 40 milligrams (“mg”), preferably in an amount about 10 to about 30 mg, more preferably in an amount of about 15 to about 20 mg. Paroxetine can be administered in the form of an aqueous solution or substantially solid dosage form. Paroxetine can be administered in the form of commercial PAXIL® tablets in dosages of a single whole tablet or half tablet or in the form of capsules.

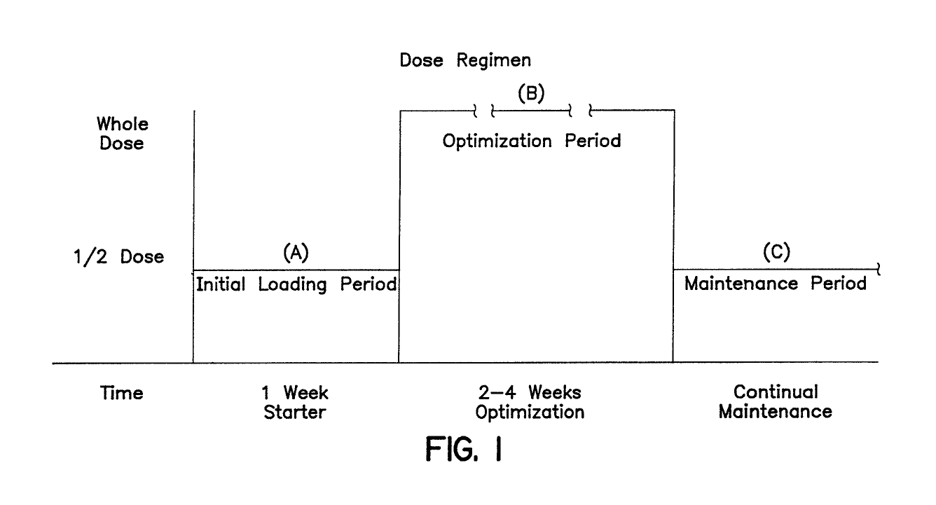

The efficacy of paroxetine administration can be optimized and maintained by following an oral therapy regimen of a brief initial loading period of daily paroxetine use, preferably followed by an optimization period of daily paroxetine use for a period of not more than four weeks prior to the patient converting to ad lib continual maintenance use. This combination oral therapy regimen is illustrated in FIG. 1.

Referring to FIG. 1, the patient first takes (A) a daily paroxetine starter dosage over an initial loading period of seven days (1 week) in an amount that is one half of a whole paroxetine dosage, relative to the dosage amount to be taken during the paroxetine optimization period (B) immediately following the initial loading period. The optimization dosage of a whole paroxetine dosage is taken daily over an optimization period of about two to about four weeks, preferably three weeks, after which a paroxetine continual maintenance period (C) follows in which the patient then converts to a continual maintenance dosage of one half of a whole paroxetine dosage, i.e., in the same amount as the paroxetine starter dosage, taken on an ad lib basis within about two to about 20 hours, preferably within about three to about four hours, prior to sexual intercourse.

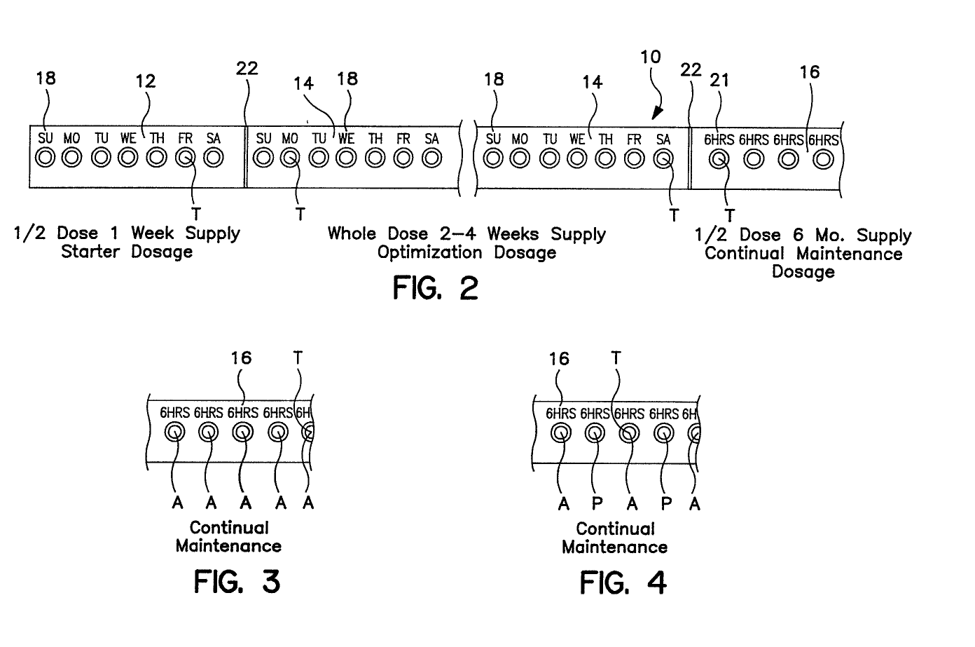

For practicing the oral therapy regimen at home, the patient is preferably given access to a kit containing a multiplicity of paroxetine dosage forms and instructional literature explaining the oral therapy regimen. One kit embodiment for practicing the oral therapy regimen illustrated in FIG. 1 is illustrated in FIG. 2 employing a tablet dosage form for convenience of discussion and not by way of limitation. Referring to FIG. 2, the kit package 10 can comprise an articulated strip of calendar cards bearing blister-packaged tablets T and arranged for a designated regimen of dosages in sequential order shown as a paroxetine starter dosage strip 12, a paroxetine optimization dosage strip 14 and a paroxetine continual maintenance dosage strip 16. Preferably, dosage day indicia 18 are provided on the paroxetine starter dosage strip 12 and on the paroxetine optimization dosage strip 14 for correlating a particular dosage to a particular day on a one to one tablet T relationship. As illustrated in FIG. 2 dosage day indicia can identify the sequential day of the week, such as Mon, Tu, We, etc. but can alternatively identify the sequential date of treatment as Day 1, Day 2, Day 3 etc. up to Day 7 on paroxetine starter dosage strip 12, and as Day 8, Day 9, Day 10, etc. up to Day 28 on the paroxetine optimization dosage strip 14. Preferably instructional indicia 21 is provided with the paroxetine maintenance dosage strip 16 for correlating a particular dosage to a particular time, illustrated as “6 Hrs” in FIG. 2 but can also be a stated instruction, such as “take after breakfast or lunch” or “take within two to four hours before sexual intercourse,” or the like. The instructional indicia can be a memory aid for recording the time interval for taking each tablet to delay the onset of ejaculation by the patient during subsequent intercourse.

The strip of calendar cards are illustrated as linear strips with an articulating hinge 22 located at seven tablet intervals for conveniently folding the kit for carrying and storage purposes. The articulating hinge 22 also can serve as a tear off hinge to separate the strips at the end of each regimen period after the dosage contained therein is used. The paroxetine starter dosage strip 12 is illustrated as containing a one week supply of tablets each of which is a ½ dose amount, relative to the whole dose amount provided by each tablet in the paroxetine optimization dosage strip 14. The paroxetine optimization dosage strip 14 can be two to four, preferably three, articulated strips, each containing a one week supply of tablets, each of which is a whole dose amount. The paroxetine maintenance dosage strip 16 can be a plurality of articulated strips adapted to contain at least a one month total supply, preferably three to nine months total supply, more preferably a six month total supply, of continual maintenance paroxetine dosage tablets or capsules, whichever dosage form is preferred. As shown in FIG. 3, every continual maintenance tablet T (or capsule) can contain an active dosage of paroxetine A in the same ½ dose amount as in each of the starter dosage forms in the starter dosage strip. Alternatively, as shown in FIG. 4, at least every other continual maintenance tablet T (or capsule) can contain an active dosage of paroxetine A in the same ½ dose amount as in the starter dosage forms in the starter dosage strip with the remaining tablets (or capsules) containing a placebo P for the purpose of ensuring better compliance of the male patient to a continual maintenance dosing regimen.

The dosage strips can be configured and adapted to contain each discrete paroxetine dosage form in sealed and linear spaced relationship from one another by any packaging methods known in the art, such as in individual blister seals as illustrated in FIG. 2, in by depressions or the like. The kit preferably includes informational literature explaining the oral therapy regimen in the form of pamphlets, flyers and the like.

It is recognized that the kit embodiment is illustrated in FIG. 2 in the form of linear dosage strips, but is not limited thereto and can be in rectangular or circular configured forms, so long as the dose regimen therapy can be practiced.

In one preferred method aspect, paroxetine can be administered to the patient according to the following oral therapy regimen:

(A) first a starter dosage of a single dosage form containing about 5 to about 40 milligrams, preferably about 10 to about 30 milligrams, more preferably about 15 to about 20 milligrams, paroxetine is taken by the patient daily over an initial loading time period of about seven days;

(B) next, an optimization dosage of a single dosage form containing twice the amount of paroxetine as the starter dosage, i.e., about 10 to about 80 milligrams, preferably about 10 to about 40 milligrams, more preferably about 15 to about 30 milligrams, paroxetine is taken by the patient daily over an optimization time period of about two to about four weeks, preferably about three weeks; and then

(C) a continual maintenance dosage of a single dosage form containing the same amount of paroxetine as the starter dosage, i.e., about 5 to about 40 milligrams, preferably about 10 to about 30 milligrams, more preferably about 15 to about 20 milligrams, paroxetine, taken within a period of about two to about 20 hours, preferably within about three to about six hours, prior to engaging in sexual intercourse.

If a starter initial loading dosage regimen of about 5 to about 40, preferably of about 10 milligrams, paroxetine sufficiently delays the onset of premature ejaculation and produces “ejaculatory recruitment”, i.e., improved ejaculatory control over pre-treatment ejaculation, the patient can choose to omit the optimization dosage period and convert to an continual maintenance dosage usage of about 5 to about 40 milligrams, preferably about 10 to about 30 milligrams, paroxetine. Alternatively, a patient can simply practice an oral therapy of continual maintenance administration by taking a single dose of paroxetine of about 5 to about 40 milligrams, preferably about 10 to about 30 milligrams, more preferably about 15 to about 30 milligrams, within several hours prior to engaging in sexual intercourse.

It was found that paroxetine delays ejaculation and prolongs the ejaculatory latency time when it is administered on a continual maintenance basis. An increase in frequency of sexual intercourse was noted as well. Even in studies which did not inventory sexual satisfaction for either the men studied or their partners, the observed improved ejaculatory control and increased frequency of intercourse suggest improved sexual response and satisfaction. Delayed ejaculation and, as a consequence, improved ejaculatory control, was observed within 1-2 weeks of initiating treatment. This acute effect was believed due to paroxetine's direct blocking effect on central serotonergic re-uptake and cannot be attributed to a decrease in psychopathology because none of the men patients were clinically depressed. Moreover, the amount of paroxetine employed was subtherapeutic for depression. Furthermore, the antidepressant side effect normally associated with paroxetine was not reported to occur within 1-2 weeks. Additionally, it is possible that some of the improved ejaculatory control and some sustained long term improvement after withdrawal of paroxetine also observed could be related to a reduction in performance anxiety due to improved patient/partner perceived sexual success.

It was found that paroxetine administered on a daily basis produced significantly more ejaculatory control in significantly more patients than continual maintenance administration. However, the efficacy of paroxetine as a continual maintenance therapy was optimized by preceding the continual maintenance administration with an initial loading period of daily acute dosing over a sufficient period to achieve ejaculatory recruitment, preferably of less than about four weeks of duration, more preferably of about one week duration.

A failure to respond to paroxetine treatment in any form of administration was found to occur more likely in patients suffering from lifelong premature ejaculation.

Patients treated solely with a regimen of continual maintenance dosage of paroxetine administration over a period of about four weeks improved in ejaculatory control from a mean ELT of less than one minute to a mean ELT of at least 1.5 minutes, an increase that was judged statistically greater (by Student's t-test comparison) than the pre-treatment ELT, though statistically less than the change in mean ELT achieved with patients who had converted to a continual maintenance regimen following an initial loading regimen. Patients converted to continual maintenance regimen following a period of four weeks initial loading and four weeks continual maintenance improved in ejaculatory control from a mean ELT of less than one minute to a mean ELT of greater than 2.5 minutes, an increase that was judged statistically greater (by Student's t-test comparison) than the pretreatment ELT.

The efficacy of a continual maintenance regimen was also observed in randomized, single blind cross-over studies with normally potent male patients suffering from premature ejaculation employing the same selection criteria described earlier. All of the patients were heterosexual, had no other sexual disorders and were either married or in a stable relationship. In crossover studies over a total of 11 weeks time, one group of patients were first treated over a four week period with paroxetine (20 mg) administered about three to about four hours before sexual intercourse, then given a three week drug-free “washout” period, and subsequently administered placebo in the same manner for a further four week treatment. A second group of patients was similarly treated, except that placebo was taken during the first four-week period and paroxetine (20 mg) was taken after the washout period during the second four week treatment.

The optimization and continual maintenance of the efficacy of paroxetine by practicing a conversion regimen was also observed over a 17 week total period in randomized, single blind cross-over studies. One group of patients followed a requested regimen of: (1) taking paroxetine (10 mg) daily for three weeks; (2) converting to taking paroxetine (20 mg) ad lib about three to four hours before sexual intercourse for a continual maintenance period of a further four weeks; (3) adhering to a drug-free washout period for three weeks; (4) taking placebo daily administered as in step (1); and (5) taking placebo administered before sexual intercourse as in step (2).

The optimization and continual maintenance of the efficacy of paroxetine was also observed by a similar conversion regimen study made with a second group of patients, in which in the above described regimen sequence steps (1) and (2) placebo administration was employed with paroxetine (10 mg) being employed in step (4) and paroxetine (20 mg) in step (5).

For efficacy studies, tablets of paroxetine and placebo tablets employed were substantially identical and indistinguishable from one another in size and appearance.

The present invention is illustrated by the following studies with participating normally potent male patients suffering from premature ejaculation dysfunction treated with paroxetine hydrochloride.

### EXAMPLE 1

### Dose Conversion Regimen and Continual Maintenance Regimen

This example illustrates the efficacy of paroxetine administered in a dose conversion regimen and in a continual maintenance regimen.

Materials and Methods

Normally potent male patients (94 total) suffering from premature ejaculation were enrolled in a prospective study to assess the efficacy and tolerance of paroxetine hydrochloride (AROPAX®) in the management of premature ejaculation. All patients in the study group were heterosexual, had no other sexual disorders and were either married or in a stable relationship. The baseline pre-treatment ELT for the group, determined as the mean of measurements from a minimum of two acts of sexual intercourse was 2.2 (range 2-3) as reported by patients prior to commencement of the study using a stop watch operated by the patients. All patients enrolled in the study had an ELT of less than one minute and were regarded as having severe premature ejaculation.

The mean age of the 94 patients was 39 years (range 18-61 years). At the start of the study, the mean pre-treatment ejaculatory latency time as measured by a Registered Nurse under clinical conditions using a stopwatch was 0.4 min. (range 0-1 min.). Vital signs, such as heart rate, blood pressure and the like were also monitored by the Registered Nurse.

The mean pre-treatment frequency of sexual intercourse of the group was 205 over a period of four weeks at 0.4 times/week. Of the group, 55 men (59%) had lifelong premature ejaculation, with the remaining 39 men (41%) describing acquired premature ejaculation with previous acceptable ejaculatory control. Of the 55 men with lifelong premature ejaculation, 10 men (18%) had severe lifelong premature ejaculation and had never achieved intravaginal ejaculation.

The patients in the study were enrolled into two groups, Group A and Group B. Group A comprised 61 patients with a mean age of 40 years (range 22-61), 37 men having lifelong premature ejaculation and the remaining 24 men having acquired premature ejaculation. Group B comprised 33 patients with a mean age of 37 years (range 18-56), 18 men having lifelong premature ejaculation and the remaining 15 men having acquired premature ejaculation. Both groups had the same mean pre-treatment ELT of 0.4 min. (range 0-1 min.) and frequency of intercourse (0.4 times/week).

The patients enrolled in Group A initially received an acute dose of paroxetine (20 mg) taken daily for four weeks (Phase 1). Those patients in Group A who responded with improved ejaculatory control in Phase 1 were then subsequently treated with a single continual maintenance dose of paroxetine (20 mg) administered about 3-4 hours prior to each sexual intercourse act (Phase 2) for a further four weeks.

The patients enrolled in Group B received only a single continual maintenance dose of paroxetine (20 mg) administered about 3-4 hours prior to each sexual intercourse act during a period of four weeks.

Each patient was supplied with an ejaculation diary and instructed to record his frequency of coitus, quality of erection and orgasm, and to measure and record their ejaculatory latency time using a stop-watch. The patients were required to attempt coitus on at least two occasions each week.

Statistical analysis: A Student's t-test was used to compare the mean ELT values before and after treatment with paroxetine for each of Groups A and B.

Results

Group A—Phase 1

In Group A during Phase 1, a total of 761 sexual intercourse acts were recorded as shown in Table 1.

TABLE 1
Results of treatment of premature ejaculation
following acute dosing (Group A - Phase 1) with paroxetine, conversion
to continual maintenance dosing with paroxetine (Group A-Phase 2) and
during continual maintenance dosing only (Group B).

| | | | mean | |
|---|---|---|---|---|
| | | mean n | Frequency | |
| | n | Intercourse | Intercourse | mean |
| Study Group | men | (4 weeks) | Times/Week | ELT (min) |
| Pre- | 94 | 205 | 0.4 | 0.4 |
| Treatment | | | | |
| Group A - | 61 | 761 | 2.4 | 4.5 |
| Phase 1 (20 | | | | |
| mg acute | | | | |
| dose) | | | | |
| Group A - | 53 | 608 | 2.6 | 3.9 |
| Phase 2 (20 | | | | |
| mg continual | | | | |
| maintenance | | | | |
| dose) | | | | |
| Group B | 33 | 298 | 2.2 | 1.5 |
| (20 mg | | | | |
| continual | | | | |
| maintenance | | | | |
| dose) | | | | |

The mean ELT for Group A during Phase 1 was 1.1 min. after one week of treatment, increasing to 1.6 min. after two weeks, 3.5 min. at three weeks and 4.5 min. at four weeks. The increase in mean ELT was judged statistically superior to pre-study levels at two, three and four weeks (all p<0.001). The mean frequency of acts of sexual intercourse was 2.4 times/week. Of the 61 patients in this Group A, 53 (87%) regarded their ejaculatory control as significantly improved, had an ELT after four weeks of treatment of 5.1 min. (range 2-17 min.) and a reported frequency of sexual intercourse acts of 2.5 times/week.

The remaining 8/61 patients regarded that they had not achieved improved control and had a mean ELT of 0.6 min. (range 0-2 min.) at four weeks and a frequency of sexual intercourse acts of 1.7 times/week. However, lifelong premature ejaculation was present in 6 men of the cohort of eight who failed to respond to daily paroxetine. The mean ELT for the 53/61 men who did regard their ejaculatory control as improved was judged statistically higher than that of the remaining 8/61 men who thought that they had not achieved improved control (p <0.001). The 53 patients in Group A—Phase 1 who achieve improved ejaculatory control then converted to continual maintenance use of paroxetine (Phase 2).

Group A—Phase 2

In Group A-Phase 2, 608 additional acts of sexual intercourse were recorded for the converted 53 patent group with a mean ELT of 3.9 min. (range 0-10 min. and a frequency of acts of sexual intercourse 2.6 times/week as shown in Table 1 after a mean follow-up of 4.4 weeks of treatment. One patient was lost to follow up. Of the patients, 36/53 (69%) regarded that they had maintained improved ejaculatory control after four weeks of additional continual maintenance dosage treatment with paroxetine, recording 391 sexual intercourse acts with a mean ELT of 5.5 min. (range 2-10) and a frequency of 2.4 acts of sexual intercourse/week. The remaining 16/53 men (31%) who regarded that they had failed to maintain improved ejaculatory control recorded 229 intercourse with a mean ELT of 0.6 min. (range 0-2 min.) at four weeks and a frequency of 2.8 acts of sexual intercourse/week. However, lifelong premature ejaculation was present in 11 men in the cohort of 16 who failed to respond to paroxetine continual maintenance treatment.

The mean ELT for Group A patients overall was judged statistically greater than that of their mean pre-study ELT (p <0.001). The mean ELT for patients in Group A-Phase 1 and Group A-Phase 2 was not judged to be statistically different. The mean ELT for the 36/53 men who reported that they had maintained improved ejaculatory control was judged statistically higher than that of the remaining 16/53 men who felt that they had not maintained improved ejaculatory control (p<0.001).

Group B

In group B, 298 acts of sexual intercourse were recorded with a mean ELT of 1.5 min (range 0-5 min) and a frequency of 2.2 acts of sexual intercourse/week after a mean follow-up of 4.1 weeks of treatment as shown in Table 1. This result was judged statistically greater than the pre-treatment ELT (p<0.05) and statistically less than the mean ELT for the patients in Group A-Phase 2 (p><0.001). Of Group B, 14/33 patients regarded their ejaculatory control as significantly improved, had a mean ELT of 2.7 min. (range 2-5 min.) and a frequency of 2.4 acts of sexual intercourse/week. This ELT was judged statistically superior to their mean pre-treatment interval (p<0.05).

The remaining 19/33 patients reported no change in ejaculatory control with a mean ELT of 0.4 min. (range 0-2 min.) and frequency of two acts of intercourse/week. However, lifelong premature ejaculation was present in 15 men in the cohort of 19 who failed to respond to continual maintenance paroxetine. The mean ELT for these patients was not judged statistically superior to their pre-study mean ELT (p>0.05).

Significantly, intra-vaginal ejaculation was achieved for the first time for 10/10 men (including four patients in Group B) having severe lifelong premature ejaculation dysfunction who had never previously achieved intra-vaginal ejaculation. Of these, 8/10 patients had previously undergone and failed to respond to one or more trials of treatment with psychosexual counseling. These patients are considered as suffering from severe and refractory premature ejaculation, and previous treatment failures. Paroxetine drug treatment salvaged them from life-long ejaculatory dysfunction and its relationship sequella.

Overall, paroxetine was reasonably well tolerated by the patients enrolled in this study as shown in Table 2 below, although acute treatment with 20 mg paroxetine/day in Group A patients during Phase 1 was associated with anejaculation in 5/61 (8%) patients and inhibition of orgasm despite achieving ejaculation in 3/61 (5%) patients.

TABLE 2
Incidence of adverse effects in patients treated for
premature ejaculation during acute dosing (Group A-Phase 1), on
conversion to continual maintenance dosing with paroxetine (20 mg)
(Group A-Phase 2) and during continual maintenance paroxetine
(20 mg) (Group B). (Some men experienced more than one adverse
effect.)

| Adverse Effect | Group A-Phase 1 | Group A-Phase 2 | Group B |
|---|---|---|---|
| Anorexia | 1 | — | — |
| Anejaculation | 5 | — | — |
| Gastrointestinal | 2 | — | — |
| Upset | | | |
| Drowsiness | 1 | — | — |
| Reduced libido | 3 | — | — |
| Inhibited orgasm | 3 | — | — |
| Total Patients | 12/61 (20%) | 0/53 | 0/33 |

One patient who experienced anejaculation declined further treatment with paroxetine, the remaining 4/5 patients achieving ejaculation with a reduced dose of 5 mg (n=3) and 10 mg (n=1). These five patients had a mean pre-study ELT of 0.9 min. which was higher than that of the entire study group (0.4 min.) and as such, one would expect a lesser incidence of anejaculation in patients with more severe premature ejaculation especially if lower doses were employed.

The occurrence of anejaculation with sertraline, another member of the SSRI class of anti-depressants, has been reported as being dose related. For example, McMahon, C. G. in “Treatment of premature ejaculation with sertraline hydrochloride: A single-blind placebo controlled crossover study,” Journal of Urology, 159, 1935 (1998) reported that while all patients in a study group of 46, treated with 25 mg sertraline/day, managed to ejaculate during sexual intercourse, 4/46 (9%) men treated with 50 mg sertraline/day and 10/46 patients (22%) treated with 100 mg sertraline/day were unable to ejaculate after prolonged sexual intercourse. No dose related correlation has been reported between paroxetine dosage and it's antidepressant effect and the incidence of adverse effects. Crenshaw et al. in U.S. Pat. No. 5,276,042 reported that in their limited clinical experience, the incidence of retarded ejaculation in patients treated with paroxetine for depression is dose related, mainly occurring at dosage amounts above 20 mg.

None of the patients taking continual maintenance paroxetine during the Group A-Phase 2 regimen, or in the Group B regimen reported any adverse effect including anejaculation as seen in Table 2. Erectile dysfunction was not reported by any of the patients.

In Group B, 14 of 33 (42%) patients treated solely with continual maintenance paroxetine achieved ejaculatory control as opposed to 53 of the 61 patients (87%) in Group A-Phase 1 who were treated with daily paroxetine. In addition, the mean ELT (5.1 min.) of the latter Group A in Phase 1 was judged statistically superior to the mean ELT of (2.7 min.) (p <0.05) of Group B patients. Clearly, paroxetine administered on a daily basis produced significantly greater ejaculatory control in a significantly greater number of patients than did continual maintenance paroxetine use. However, the efficacy of use of continual maintenance paroxetine was optimized by an initial acute dosing period.

Of the 53 patients in Group A-Phase 2 converted to treatments with continual maintenance paroxetine after an initial daily administration period of four weeks, 36 (68%) patients reported sustained ejaculatory control with a mean ELT of 5.5 min. which was not significantly different from that achieved during the initial daily dosing phase (5.1 min.). Therefore, overall, 59% of the patients in Group A (36/61) achieved and maintained improved ejaculatory control with treatment of premature ejaculation with a combination regimen of an initial acute dosing period of daily paroxetine followed by conversion to continual maintenance paroxetine use.

The observation that an initial loading with paroxetine sufficient to produce “ejaculatory recruitment” may be related to the non-linear pharmacokinetics of paroxetine. As a result of first-pass metabolism which is almost exclusively mediated by the P450 2D6 enzyme, the amount of paroxetine available to the systemic circulation is known to be less than that absorbed from the gastrointestinal tract. However, paroxetine is also a potent inhibitor of this enzyme, thereby effectively inhibiting it's own metabolism and demonstrating non-linear pharmacokinetics. Therefore, as paroxetine concentration increases with multiple dosing, the P450 2D6 activity decreases thus prolonging drug clearance and resulting in a disproportionately greater increase in its concentration with every dose.

On the basis of these results, an initial load dosing treatment with daily paroxetine for four weeks followed by continual maintenance paroxetine administration was the preferred approach in that it offered 59% of the patients improved ejaculatory control with minimal adverse effects. However, following conversion from daily acute dosing to chronic paroxetine use, a considerable number of those men who initially responded to daily paroxetine (32% in this study) had recurrence of premature ejaculation prompting a return to continuing daily acute paroxetine usage. However, the sole use of continual maintenance paroxetine administration was effective in 42% of the patient group and was most appropriate in those men troubled by the adverse effects of daily paroxetine particularly anejaculation or retarded ejaculation. An ad lib continued maintenance paroxetine regimen has particular appeal for those patients who have less severe premature ejaculation, have sexual intercourse infrequently and/or prefer to avoid taking daily medication.

In conclusion, paroxetine appears to be a useful and reasonably well tolerated oral therapy regimen for treating premature ejaculation with improved ejaculatory control usually occurring within 1-2 weeks and subsequent increased frequency of sexual intercourse. Administration of continual maintenance paroxetine improved ejaculatory control and was more efficacious when preceded by an initial acute dosing period.

### EXAMPLE 2

### Cross-Over Regimen

This example illustrates and compares the efficacy of paroxetine administered in a continual maintenance dosage regimen and in a randomized cross-over conversion to a continual maintenance regimen against that of a similarly randomized cross-over placebo regimen. The placebo tablets were substantially identical in size and appearance to the active drug tablet.

Materials and Methods

Normally potent male patients (64 total) suffering from premature ejaculation (“PE”) (ejaculation within one minute of vaginal intromission) were enrolled into one of two randomized single blind cross-over comparative studies of paroxetine hydrochloride (AROPAX®, SmithKline Beecham Pharmaceuticals) and placebo. All patients in the study group were heterosexual, had no other sexual disorders and were either married or in a stable relationship. Patients with erectile dysfunction, reduced sexual desire, inhibited male orgasm, chronic depressive, psychiatric or physical illness, alcohol or substance abuse and the use of psychotropic and anti-depressant medication were excluded from the trial.

The patients were divided into two study groups, Group 1 and Group 2. The patient characteristics of each study group are shown in Table 3.

TABLE 3
Patient characteristics of Study Groups 1 and 2

| | Study Group 1 | Study Group 2 |
|---|---|---|
| n patients | 26 | 42 |
| n (primary PE) | 19 | 32 |
| n (secondary PE) | 7 | 10 |
| n (never ejaculated intravaginal) | 3 | 5 |
| Mean age | 39.5 years | 40.5 years |
| Mean Baseline ELT | 0.3 min. | 0.5 min. |

Study Group 1

Study Group 1 was comprised of 26 patients who were randomized into two treatment groups, Group A and Group B. Group A received a continual maintenance paroxetine (20 mg) dosage administered ad lib about 3-4 hours before sexual intercourse over a period of four weeks. Group B received placebo similarly administered about 3-4 hours before sexual intercourse. Patients were reviewed every two weeks. Cross-over was then conducted in both groups after four weeks of treatment, following an intervening three week drug free “washout” period, for a further four weeks of treatment.

Study Group 2

Study 2 was comprised of 42 patients who were randomized into two treatment groups, Group C and Group D. Group C received a regimen of (1) a paroxetine (10 mg) daily for three weeks; (2) conversion to a continual maintenance paroxetine (20 mg) dosage administered ad lib about 3-4 hours before sexual intercourse for a further four weeks; (3) a three week drug-free washout period; (4) placebo daily for three weeks; and (5) conversion to administration of placebo about 3-4 hours before sexual intercourse. Group D received a regimen of (1) placebo administered daily for three weeks; (2) conversion to placebo administered about 3-4 hours before sexual intercourse for a further four weeks; (3) a three week drug-free washout period; (4) paroxetine (10 mg) daily for three weeks; and (5) conversion to a continual maintenance paroxetine (20 mg) dosage administered ad lib about 3-4 hours before sexual intercourse. Patients were monitored every two weeks.

Paroxetine at 10 mg is a subtherapeutic dose for the treatment of depression so that onset of delayed ejaculation is more likely to be directly due to the central serotonergic effect of paroxetine and not simply an improvement in psychopathology.

None of the patients received any formal psychosexual counseling. Patients were supplied with a diary and were asked to record their frequency of coitus, quality of erection and orgasm. Patients were asked to measure their ELT using a stopwatch. Pre-treatment mean ELT was measured over a three week baseline period during which patients were asked to have sexual intercourse at least three times. Pre-treatment frequency of coitus was the mean number of attempts in the previous three months. Patients were asked not to use condoms or topical penile anaesthetic creams or sprays.

The frequency data of coitus and ejaculatory latency time (ELT) during the paroxetine and placebo treatment phases were statistically compared (Student's t-test).

Results

Study Group 1

The mean age of the 26 male patients in Study Group 1 was 39.5 years (range 19-55), the pre-treatment mean ELT was 0.3 minutes with a range of ejaculation occurring from foreplay or at intromission, to 60 seconds after intromission as shown in Table 4.

In both Group A and Group B, the mean pre-treatment frequency of coitus was 0.5 per week and the mean pretreatment ELT was 0.3 min. In Group A, both the ELT and frequency of coitus respectively increased during the initial continual maintenance paroxetine treatment to 3.2 min., and 3.2/week at week 4 and fell respectively during the subsequent placebo treatment to 0.45 min., and 0.9/week—week 11. In Group B, the ELT and frequency of coitus increased slightly during initial placebo treatment respectively to 0.6 min., and 1.3/week at week 4 but increased dramatically during subsequent paroxetine treatment to respectively 3.5 min., and with continual maintenance to 3.1/week by week 11. The ELT in Groups A and B during treatment with continual maintenance paroxetine was judged statistically superior to that the placebo at two, three and four weeks (p <0.001) as shown in Table 4 below.

TABLE 4
Mean frequency of coitus and the ejaculatory latency
times from 1 to 11 weeks in 26 patients during continual maintenance
paroxetine, drug-free washout and placebo treatment phases.

| | |
|---|---|
| Study Group 1-A (13 patients) | Study Group 1-B (21 patients) |

| Treatment | | Coitus | Ejaculatory | | Coitus | Ejaculatory |
|---|---|---|---|---|---|---|
| Duration | Treatment | Frequency/ | Latency Time | Treatment | Frequency/ | Latency |
| (Wks) | Phase | wk | (min.) | Phase | wk | Time (min.) |
| Pre- | — | 0.5 | 0.3 | — | 0.5 | 0.3 |
| treatment | | | | | | |
| 1 | Paroxetme | 1.1 | 1.0 | Placebo | 1.5 | 0.4 |
| | 20 mg | | | | | |
| 2 | Paroxetine | 1.4 | 3.0 | Placebo | 1.7 | 0.7 |
| | 20 mg | | | | | |
| 3 | Paroxetine | 2.3 | 2.9 | Placebo | 1.0 | 0.7 |
| | 20 mg | | | | | |
| 4 | Paroxetine | 3.2 | 3.2 | Placebo | 1.3 | 0.6 |
| | 20 mg | | | | | |
| 5 | Washout | 3.0 | 2.0 | Washout | 1.1 | 0.7 |
| 6 | Washout | 2.3 | 2.1 | Washout | 0.7 | 0.5 |
| 7 | Washout | 2.5 | 1.5 | Washout | 0.8 | 0.5 |
| 8 | Placebo | 1.2 | 1.3 | Paroxetme | 0.6 | 1.3 |
| | | | | 20 mg | | |
| 9 | Placebo | 1.3 | 0.5 | Paroxetine | 1.9 | 2.8 |
| | | | | 20 mg | | |
| 10 | Placebo | 1.5 | 0.35 | Paroxetine | 3.5 | 3.5 |
| | | | | 20 mg | | |
| 11 | Placebo | 0.9 | 0.45 | Paroxetine | 3.1 | 3.5 |
| | | | | 20 mg | | |

The ELT values for the patients in both Groups C and D during daily paroxetine treatment were judged statistically superior to daily placebo at two (p<0.05) and at three weeks (p<0.001) of treatment. The ELT during continual maintenance paroxetine treatment was judged statistically higher than daily placebo (p<0.001). The ELT during continual maintenance paroxetine treatment was judged statistically higher than daily administration of paroxetine at week seven for Group C (p<0.05) and at week 17 for Group D (P<0.001).

The mean ELT during the continual maintenance paroxetine phase of study 2 was greater than the mean ELT during the continual maintenance paroxetine phase of study Group 1 (p <0.05) suggesting that the ejaculatory control achieved with continual maintenance paroxetine is significantly better if patients are initially treated with a daily paroxetine load.

Intra-vaginal ejaculation was achieved for the first time with paroxetine by one of three patients who had never previously achieved it. The age of this man was 26.5 years and intra-vaginal ejaculation was achieved after three attempts at sexual intercourse and after two weeks of treatment with paroxetine. He did not achieve intra-vaginal ejaculation with placebo. None of the patients reported any adverse effects with continual maintenance use of paroxetine or with placebo.

Study Group 2

The mean age of the 42 patients in Study Group 2 was 40.5 years (range 20-51) and the pre-treatment mean ELT was 0.5 minutes with a range of ejaculation occurring from during foreplay or at intromission, to 60 seconds after intromission as shown in Table 5. The mean frequency of coitus before treatment was 0.6 times per week in Group C and 0.5 times per week in Group D.

In Group C, both the mean ELT and mean frequency of coitus increased during continual maintenance paroxetine treatment respectively to 4.3 min., and 2.7/week at week 3 and were maintained during subsequent continual maintenance paroxetine treatment respectively at 5.8 min., and 2.4/week at week 7. Both parameters fell respectively during subsequent administration of placebo to 0.9 min., and 1.4/week at week 13, falling further during final conversion to continual maintenance with placebo to 0.6 min., and 0.4/week at week 17.

In Group D, both the mean ELT and frequency of coitus respectively increased slightly during the initial placebo treatment to 0.8 min., and 1.3/week at week 3 and during the continual maintenance with placebo phase to 1.1 min., and 0.3/week at week 7 but increased dramatically during subsequent continual maintenance paroxetine treatment to 3.3 min., and 3.1/week at week 13, increasing still further during the final continual maintenance paroxetine treatment to 6.1 min., and 2.7/week by week 17 as shown in Table 5 below.

TABLE 5
Mean frequency of coitus and the mean ejaculatory
latency times over a period of 1 to 17 weeks in 42 patients during
paroxetine, drug-free washout periods and placebo treatments.

| | |
|---|---|
| Study Group 2-C (21 patients) | Study Group 2-D (21 patients) |

| Treatment | | Coitus | Ejaculatory | | Coitus | Ejaculatory |
|---|---|---|---|---|---|---|
| Duration | Treatment | Frequency/ | Latency Time | Treatment | Frequency/ | Latency |
| (Wks) | Phase | wk | (min.) | Phase | wk | Time (min.) |
| Pre- | — | 0.6 | 0.5 | — | 0.5 | 0.5 |
| treatment | | | | | | |
| 1 | Paroxetine | 1.7 | 1.2 | Placebo | 1.1 | 0.7 |
| 2 | Paroxetine | 1.9 | 1.8 | Placebo | 1.2 | 0.5 |
| 3 | Paroxetme | 2.7 | 4.3 | Placebo | 1.3 | 0.8 |
| 4 | Paroxetine | 2.3 | 4.5 | Placebo | 1.0 | 0.7 |
| | (c.m.) | | | (c.m.) | | |
| 5 | Paroxetine | 2.0 | 5.2 | Placebo | 0.6 | 1.0 |
| | (c.m.) | | | (c.m.) | | |
| 6 | Paroxetine | 2.9 | 4.7 | Placebo | 0.4 | 0.9 |
| | (c.m.) | | | (c.m.) | | |
| 7 | Paroxetme | 2.4 | 5.8 | Placebo | 0.3 | 1.1 |
| | (c.m.) | | | (c.m.) | | |
| 8 | Washout | 2.7 | 2.1 | Washout | 0.5 | 0.9 |
| 9 | Washout | 2.2 | 1.1 | Washout | 0.5 | 0.4 |
| 10 | Washout | 1.5 | 1.2 | Washout | 0.7 | 0.5 |
| 11 | Placebo | 1.5 | 1.3 | Paroxetine | 2.0 | 2.2 |
| 12 | Placebo | 1.2 | 1.5 | Paroxetine | 2.2 | 3.5 |
| 13 | Placebo | 1.4 | 0.9 | Paroxetine | 3.1 | 3.3 |
| 14 | Placebo | 1.0 | 0.6 | Paroxetine | 3.0 | 4.2 |
| | (c.m.) | | | (c.m.) | | |
| 15 | Placebo | 0.5 | 0.4 | Paroxetine | 2.6 | 3.9 |
| | (c.m.) | | | (c.m.) | | |
| 16 | Placebo | 0.5 | 0.3 | Paroxetine | 2.5 | 4.6 |
| | (c.m.) | | | (c.m.) | | |
| 17 | Placebo | 0.4 | 0.6 | Paroxetine | 2.7 | 6.1 |
| | (c.m.) | | | (c.m.) | | |

c.m. = continual maintenance
Weeks 1-3 and 11-13 = 10 mg; Weeks 4-7 and 14-17 = 20 mg

Intra-vaginal ejaculation was achieved for the first time with paroxetine in all of the five patients who had never previously achieved it. The mean age of these men was 34.5 years and intra-vaginal ejaculation was achieved after a mean of four attempts at sexual intercourse and after two weeks of treatment with paroxetine. Three of these five patients were in Study Group 1-C and ⅔ achieved intra-vaginal ejaculation during the subsequent placebo treatment phase. The remaining two patients were in study Group 2-D and did not achieve intra-vaginal ejaculation during the initial placebo treatment phase. One of these five patients reported anorexia with paroxetine.

Paroxetine appeared to be well tolerated by the patients enrolled in Study Group 2 . As shown in Table 6, adverse effects were minor and occurred with daily acute doses of paroxetine or placebo.

TABLE 6
Incidence of adverse effects from 1-17 weeks in
Study Group 2 during paroxetine, placebo and washout treatment phases.

| | Daily | C.M.* | Daily | C.M.* |
|---|---|---|---|---|
| Adverse effect | Paroxetine | Paroxetine | Placebo | Placebo |
| Anorexia | 1 | — | — | — |
| Anejaculation | 3 | — | — | — |
| Gastrointestinal | 3 | — | — | — |
| upset | | | | |
| Reduced Libido | 2 | — | — | — |
| Erectile Dysfunction | — | — | 2 | — |
| Headaches | — | — | — | 1 |
| Total Patients | 7/42 (17% | — | 2 (5%) | 1 (2.4%) |

*C.M. = continual maintenance treatment phase

Three patients taking paroxetine daily experienced anejaculation and withdrew from the study. However, one of these three patients had primary erectile dysfunction; the remaining two patients describing previous acceptable ejaculatory control. Two of this group had a pre-treatment ELT (0.9 and 1 min.) which was higher than the mean for Study Group 2 (0.5 min.). Withdrawal of the drug effected return of ejaculation within 2-10 days with one patient subsequently responding to a lower dose of paroxetine (5 mg) with good ejaculatory control.

None of the three patients who experienced adverse effects of anorexia, drowsiness or gastrointestinal upset, regarded them as disabling and none withdrew from the study. Other adverse sexual effects typically reported with tricyclic and SSRI class antidepressants when used in the treatment of depression such as erectile dysfunction, reduced intensity of orgasm or inhibited orgasm were not reported by the patients enrolled in this study. Two patients did however describe reduced sexual desire despite achieving good ejaculatory control. The lack of other sexual adverse effects is possibly related to the low dose of paroxetine used in this study compared to the higher doses of paroxetine therapeutically used for the treatment of depression. The development of erectile dysfunction in two patients taking daily placebo possibly related to worsening performance anxiety due to a lack of response from placebo. The subsequent return of spontaneous erections during the active drug phase of the study in one patient is consistent with this.

Study Group 1 demonstrated that continual maintenance paroxetine administration to patients with premature ejaculation delayed the onset of ejaculation by prolonging the ejaculatory interval (ELT) within 1-2 weeks of treatment. This beneficial effect was significantly superior to that achieved with continual maintenance placebo after two weeks of treatment. Study Group 2 demonstrated that the improved ejaculatory control achieved with an initial loading of daily acute administration of paroxetine was maintained on conversion to continual maintenance paroxetine usage.

Furthermore, Study Groups 1 and 2 both demonstrated that the ejaculatory control achieved with use of continual maintenance paroxetine can be optimized if patients follow a regimen of being initially treated with daily acute dosages of paroxetine prior to conversion to continual maintenance paroxetine dosages. Although the mean ELT (3.2 min.) of patients taking continual maintenance paroxetine dosages was greater than that of patients taking placebo (0.45 min.) in both Groups 1-A and 1-B, it was significantly less than that achieved with continual maintenance paroxetine after an initial loading with paroxetine administered daily (5.8 min.). As a result of first-pass metabolism which is almost exclusively mediated by the P450 2D6 enzyme, the amount of paroxetine available to the systemic circulation is known to be less than that absorbed from the gastrointestinal tract. However, paroxetine is also a potent inhibitor of this enzyme, thereby effectively inhibiting its own metabolism and demonstrating non-linear pharmacokinetics. Therefore, as paroxetine concentration increases with multiple dosing, the P450 2D6 activity decreases thus prolonging drug clearance and resulting in a disproportionately greater increase in its concentration with every dose. This may explain the “ejaculatory recruitment” that occurs in patients treated with continual maintenance paroxetine after initial acute paroxetine dosing.

Prolongation of the ELT within a treatment of 1-2 weeks suggests that this acute effect is due to direct blocking of central serotonergic re-uptake by paroxetine. The improved ejaculatory control observed cannot be attributed to a decrease in psychopathology since none of the patients were clinically depressed and the antidepressant effect of paroxetine has not been reported to occur with 1-2 weeks. In addition, there is no published or anecdotal data to suggest that depression can be treated with administration of paroxetine continual maintenance or any other anti-depressant medication.

The higher frequency of sexual intercourse by patients treated with either daily or continual maintenance paroxetine administration compared to that noted with placebo, was judged statistically superior after three weeks of treatment. Although this study did not employ an inventory of sexual satisfaction for either the patients studied or their partners, the observed improved ejaculatory control and increased frequency of intercourse suggested improved sexual response and satisfaction.

Six of the eight patients with severe primary premature ejaculation who achieved intravaginal ejaculation for the first time in their sexual life with treatment with paroxetine had previously undergone and failed to respond to one or more trials of treatment with psychosexual counseling. Three patients in this group were either married or were involved in a long term stable relationship.

In conclusion, paroxetine was found to be a useful and well tolerated oral treatment for premature ejaculation when administered in an oral therapy regimen of solely continual maintenance dosages or in combination with a preliminary daily initial loading period prior to conversion to continual maintenance usage. The use of continual maintenance paroxetine as a treatment for premature ejaculation was both efficacious and associated with non-adverse effects in this study. The lack of adverse effects with continual maintenance paroxetine usage may be associated with improved patient acceptance and compliance. The efficacy of a continual maintenance paroxetine regimen appears to be optimized by an initial loading period of daily paroxetine.

### EXAMPLE 3

### Three-Way Crossover Regimen

A double-blind, randomized, placebo-controlled, three-way crossover safety and efficacy study was conducted at four clinical sites, A-D. To achieve the goal of having 48 evaluable male patients, approximately 70 male patients were randomized to receive two treatments of paroxetine hydrochloride, i.e., 20 mg paroxetine capsules, 30 mg paroxetine capsules, and one treatment with placebo capsules. The study consisted of a screening period, a baseline period, and three treatment periods, separated by two washout periods. Patients who completed the study participated for about twenty-five weeks.

The patients selected were healthy men between the ages of 18 and 65 years with a diagnosis of premature ejaculation (PE). Patients who met the selection criteria provided a medical history and submitted to psychosexual and physical examinations at screening. The efficacy of the paroxetine capsules was assessed at each visit by questionnaire. Safety was assessed by physical examinations and adverse experience (AE) reports, if any, were collected throughout the course of the study. A post-treatment physical assessment was performed at the final visit.

Qualified patients who were admitted to the study were assigned an identification number in sequential order by enrollment. The patients were randomized to receive placebo and one of the two active paroxetine treatments during each crossover treatment period so that each patient would receive all treatments in random order. There were about twenty-three patients per sequence.

Selection Criteria

The patients were selected based upon the following inclusion criteria.

1. Patient and female partner must be able to understand and sign a written informed consent prior to entering into the study.

2. Heterosexual male age 18-65 years old.

3. Life long history of PE and/or history of PE within the last six months having ≦one minute ejaculation and a perception of lack of control.

4. Married or have a stable partner.

5. Agreement to attempt at least one (1) vaginal intercourse once every week (with spouse or stable partner).

6. Patient and partner, if of child-bearing age, must be willing to use an approved method of contraception throughout the entire study.

7. Patient and partner must be willing to comply with study procedures.

The patients were excluded on the basis of the following exclusion criteria.

1. Patients had evidence of other DSM-IV sexual disorder.

2. Major Affective Disorders—Schizophrenia, depression, etc.

3. Cardiac or vascular disease, hepatic, renal or pulmonary disease, diabetes, neuropathy, etc.

4. Evidence of any medical condition that would interfere with PE such as patient taking any of the following medications within the stated washout period:

| | Washout |
|---|---|
| | Period |
| Medication/Treatment | (Days) |
| Over the counter cough/cold preparation | 7 |
| Antiepileptics (e.g. phenytoin) | 30 |
| Antispasmodics (e.g. procyclidine | 30 |
| Barbiturates (e.g. phenobarbital) | 30 |
| Cimetadine | 30 |
| Other Investigational Drugs | 30 |
| Prescription or over the counter diet | 30 |
| medications or treatments | |
| Sedating or non-sedating antihistamines | 30 |
| Warfarin-like compounds (e.g. COUMADIN) | 30 |
| Lithium | 60 |
| Selective Seratonin Reuptake Inhibitors | 60 |
| (SSRIs) (e.g. ZOLOFT, PAXIL, PROZAC) | |
| Tricyclic Antidepressants (e.g. Coxefen) | 60 |
| Nortriptylene, Amitriptyline) | |
| Monoamine Oxidase Inhibitors (MAOIs) | 90 |
| (NARDIL, PARNATE, etc.) | |

Note:
1) Interactions with some antihistamines and paroxetine HCI have been associated with cardiovascular side effects, therefore, use of these drugs were prohibited in this study.
2) Interactions of MAOI and paroxetine HCI have been reported to cause serious, sometimes fatal, reactions (including hyperthennia, rigidity, myoclonus, autonomic instability with possible rapid fluctuations of vital signs, and mental status changes that include extreme agitation progression to delirium and coma). Patients taking MAOI's were excluded from the study unless washed out for 90 days.
3) Paroxetine has been shown to interact with seratonin uptake inhibitors and warfarin. The metabolism of paroxetine may be affected by co-administration with Cimetadine or phenobarbital or phenytoin.

5. Patient had history of drug/substance abuse or history of alcohol abuse within the last six months. (Patients were asked to refrain from alcohol consumption for the duration of the study).

6. Anorgasmic partner.

7. Patient or partner unable or unwilling to comply with study procedures.

8. Partner's use of fertility drugs (like CLOMED) within a 30-day washout period.

The oral paroxetine capsules were supplied in 20 mg and 30 mg dosages. Identically matching placebo capsules were also supplied. Blinding, packaging and labeling was such that each strength of paroxetine capsule and placebo capsule was indistinguishable from one another on the basis of size, appearance, shape, weight, taste, odor finish, and dissolution characteristics.

The capsules were provided in high density polyethylene, opaque, white bottles with pharmaceutically acceptable closure and foil seal (within the bottle) and 21 capsules were contained per bottle. Each bottle was appropriately identified with, among other things, the protocol number, treatment and visit numbers, patient's identification number, instructions for use and storage conditions, if necessary, and the usual cautionary warning “Caution: New Drug—Limited by Federal Law to investigational use only.” Labels were color-coded to identify each treatment (Treatments 1, 2, and 3).

Each patient received one of three treatments: 20 mg paroxetine, 30 mg paroxetine or placebo according to randomization during each crossover treatment period. The patient was instructed to take one capsule (of either placebo or active drug) orally each day during each 6-week treatment period. The capsules were to be taken within 30-60 minutes after breakfast.

A three-week washout (no treatment) period took place between the end of Treatment Period 1 and the start of Treatment Period 2, and between the end of Treatment Period 2 and the start of Treatment Period 3. The following methodology for the study was employed.

### Methodology

1. Screening Period: Visit 1 (Week -2, Day -20)

a) Obtain signed informed consent from patients and partners.

b) Review all inclusion and exclusion criteria and complete case report form.

c) Review prior and concomitant medications and record information. Ensure that patients are properly washed out of excluded medications.

d) Obtain medical history, sexual history and perform physical examination of patients.

e) Perform venipuncture on patient to obtain approximately 14 cc blood for complete blood count (CBC) and blood chemistry. Record results.

f) Conduct sexual and psychosocial assessment of patient.

g) Schedule an appointment for patients and partners to return for next visit.

2. Baseline Period: Prior to Treatment, Visit 2 (Week 1, Day 1)

a) Review selection criteria to ensure that patients are eligible to participate.

b) Assign patient number to eligible patients according to randomization.

c) Obtain baseline sexual experience.

3. Treatment Period 1: Visit 2 (Weeks 1-3, Days 1-21)

a) Provide patients with one bottle containing three weeks' supply of study medication according to randomization.

b) Instruct patients and partners to attempt sexual intercourse at least once every week. Provide patients with diaries containing questionnaires and other records. Instruct patients and partners to complete a set of questionnaires at the end of the 3-week period.

c) Instruct patients to record all concomitant medications and adverse events during the first three weeks of Treatment Period 1.

d) Remind patients to return any unused study medication, container and to bring the diaries on the next visit.

e) Schedule an appointment for patients and partners to return for Visit 3.

4. Treatment Period 1: Visit 3 (Weeks 4-6, Days 22-42)

a) Collect all unused study medication, container, and all diaries.

b) Review adverse experiences and concomitant medications during the first three weeks of Treatment Period 1. Ensure all documents are complete, including the questionnaires.

c) Provide patients with one bottle containing three weeks' supply of study medication according to randomization.

d) Instruct patients and partners to attempt sexual intercourse at least once every week. Provide patients with diaries containing questionnaires and other records. Instruct patients and partners to complete a set of questionnaires at the end of the 3-week period.

e) Instruct patients to record all concomitant medications and adverse events during this period.

f) Remind patients to return any unused study medication and container and to bring the diaries on the next visit.

g) Schedule an appointment for patients and partners to return for Visit 4.

5. Washout Period 1: Visit 4 (Weeks 7-9, Days 43-63)

a) Collect all unused study medication, container, and all diaries.

b) Conduct a brief physical examination of patients.

c) Review adverse experiences and concomitant medications recorded since the last visit. Ensure all documents are complete, including the questionnaires.

d) Instruct patients and partners to attempt sexual intercourse at least once every week. Provide patients with diaries containing questionnaires and other records. Instruct patients and partners to complete a set of questionnaires at the end of the 3-week period.

e) Remind patient to record any adverse experience, to record any concomitant medication, and to avoid excluded medications during the 2-week washout period.

f) Remind patient to bring the diaries on the next visit.

g) Schedule an appointment for patients and partners to return for Visit 5.

6. Treatment Period 2: Visit 5 (Weeks 10-12, Days 64-84)

a) Review selection criteria and ensure that the patients and partners still remain eligible.

b) Review adverse events and concomitant medications recorded since last visit. Ensure all documents are complete, including the questionnaires.

c) Provide patients with one bottle containing three weeks' supply of study medication according to randomization.

d) Instruct patients and partners to attempt sexual intercourse at least once every week. Provide patients with diaries containing questionnaires and other records. Instruct patients and partners to complete a set of questionnaires at the end of the 3-week period.

e) Instruct patients to record all concomitant medications and adverse events during this period.

f) Remind patients to return any unused study medication and container and to bring the diaries on the next visit.

g) Schedule an appointment for patients and partners to return for Visit 6.

7. Treatment Period 2: Visit 6 ( Weeks 13-15, Days 85-105)

a) Collect all unused study medication, container, and all diaries.

b) Review adverse experiences and concomitant medications since the last visit. Ensure all documents are complete, including the questionnaires.

c) Provide patients with 3 weeks' supply of study medication according to randomization.

d) Instruct patients and partners to attempt sexual intercourse at least once every week. Provide patients with diaries containing questionnaires and other records. Instruct patients and partners to complete a set of questionnaires at the end of the 3-week period.

e) Instruct patients to record all concomitant medications and adverse events during this period.

f) Remind patients to return any unused study medication and container and to bring the diaries on the next visit.

g) Schedule an appointment for patients and partners to return for Visit 7.

8. Washout Period 2: Visit 7 Weeks 16-18, Days 106-126)

a) Collect all unused study medication, container, and all diaries.

b) Conduct a brief physical examination of patients.

c) Review adverse experiences and concomitant medications since the last visit. Ensure all documents are complete, including the questionnaires.

d) Instruct patients and partners to attempt sexual intercourse at least once every week. Provide patients with diaries containing questionnaires and other records. Instruct patients and partners to complete a set of questionnaires at the end of the 3-week period.

e) Remind patient to record any adverse experience, to record any concomitant medication, and to avoid excluded medications during the 3-week washout period.

f) Remind patients to bring the diaries on the next visit.

g) Schedule an appointment for patients and partners to return for Visit 8.

9. Treatment Period 3: Visit 8 (Weeks 19-21, Days 127-147)

a) Review selection criteria and ensure that the patients and partners still remain eligible.

b) Review adverse events and concomitant medications recorded since the last visit. Ensure all documents are complete, including the questionnaires.

c) Provide patients with one bottle containing three weeks' supply of study medication according to randomization.

d) Instruct patients and partners to attempt sexual intercourse at least once every week. Provide patients with diaries containing questionnaires and other records. Instruct patients and partners to complete a set of questionnaires at the end of the 3-week period.

e) Instruct patients to record all concomitant medications and adverse events during this period.

f) Remind patients to return any unused study medication, container, and bring the diaries to the next visit.

g) Schedule an appointment for the patients and partners to return for Visit 9.

10. Treatment Period 3: Visit 9 (Weeks 22-24, Days 148-168)

a) Collect all unused study medication, container, and all diaries.

b) Review adverse experiences and concomitant medications since the last visit. Ensure all documents are complete, including questionnaires.

c) Provide patients with 3 weeks' supply of study drug according to randomization.

d) Instruct patients and partners to attempt sexual intercourse at least once every week. Provide patients with diaries containing questionnaires and other records. Instruct patients and partners to complete a set of questionnaires at the end of the 3-week period.

11. Final/Exit Visit: Visit 10 (Week 25, Days 169-175)

a) Conduct physical examination of patients.

b) Obtain 14 ml blood from patients for CBC and blood chemistries. Record results.

c) Collect all unused study mediation, container, and all diaries. Review diaries for concomitant medication use and adverse experiences. Ensure all documents, including the questionnaires, are complete.

d) Discharge patients and partners from the study.

A study summary and preliminary data are shown in Tables 7 and 8, respectively.

TABLE 7
Study Summary of Placebo and 20 and 30 mg
Paroxetine Capsules

| | | | |
|---|---|---|---|
| | | | Reason for |
| | | | Early |
| Clinical | Patients | Patients | Termination |

| Site | Enrolled | Completed | AE* | Other |
|---|---|---|---|---|
| A | 10 | 8 | 0 | 2 |
| B | 9 | 6 | 0 | 3 |
| C | 15 | 4 | 2 | 9 |
| D | 19 | 11 | 3 | 5 |
| TOTALS | 53 | 29 | 5 | 19 |

*AE = Adverse Experience

TABLE 8
Preliminary Data**
Male Baseline Latency ≦ 2 Minutes

| | | | | 20 mg | 30 mg |
|---|---|---|---|---|---|
| | Reported | | | capsule | capsule |
| | By | Baseline | Placebo | Paroxetine | Paroxetine |
| Mean Time | Patient | 1.1 | 1.6 | 6.2 | 7 |
| in minutes | | (0.2-2) | (0.3-3.1) | (0.5-20) | (0.25-45) |
| (Range) | Partner | 2 | 3.3 | 6.5 | 6.8 |
| | | (03.8.5) | (0.3-10) | (0.4-20) | (0.3-30) |
| Mean % | Patient | — | 45 | 464 | 536 |
| Change | Partner | — | 65 | 225 | 240 |
| from | | | | | |
| Baseline | | | | | |
| n | Patient | (n = 31) | (n = 24) | (n = 28) | (n = 27) |
| | Partner | (n = 29) | (n = 24) | (n = 26) | (n = 25) |

**Three subjects' data removed due to placebo latency time of 8 mm or greater

### EXAMPLE 4

### Double-Blind Regimen

A double-blind, randomized, parallel safety and efficacy study was conducted at six clinical sites, A-F. To achieve a goal of having 108 evaluable male patients, approximately 18 patients at each site were randomized for studying the efficacy of three paroxetine hydrochloride treatments employing 6 mg, 9 mg, and 12 mg paroxetine-containing capsules. The study consisted of a screening period, baseline period, and two treatment periods. The patients who completed the study participated for about nine weeks.

The patients selected were healthy men between the ages of 21 and 65 years with a diagnosis of premature ejaculation (PE). Patients who met the selection criteria provided a medical history and submitted to psychosexual and physical examinations at screening. The efficacy of the paroxetine capsules was assessed at each visit. Safety was assessed by physical examinations and adverse experience (AE) reports, if any, were collected throughout the study. A post-treatment physical assessment was performed at the final visit.

Qualified patients who were admitted to this study were assigned an identification number in sequential order by enrollment. The patients were randomized to receive one of the three active paroxetine treatments during two treatment periods. There were a total of about eighteen patients per dose/per site.

Selection Criteria

The patients were selected upon the same inclusion criteria described in Example 3, except that the age of the patients was 21-65 years and patients had a life long history of PE and/or history of PE within the last six months based on having ≦two minutes ejaculation with penetration and a perception of lack of control.

The same selection criteria for excluding a patient described in Example 3 were used.

The oral paroxetine capsules were supplied in 3 mg, 6 mg, and 9 mg dosages. The protocol of Example 3 for blind packaging and labeling was followed so that each strength of paroxetine capsule was indistinguishable from one another on the basis of size, appearance, shape, weight, taste, odor finish, and dissolution characteristics.

Each patient received one of the three paroxetine treatments: 6 mg, 9 mg, or 12 mg according to randomization. The patient was instructed to take one capsule of active drug orally each day for 6 weeks during the treatment period. The capsule was to be taken one hour after breakfast or within two hours of awakening. If a patient missed a dose, they were instructed to note this and continue with the next scheduled dose and to not take 2 capsules the next day.

The following methodology for the study was employed.

### Methodology

1. Screening Period: Visit 1 (Week -2, Day -20) The same methodology procedure described in the Screening Period of Example 3 was followed.

2. Baseline Period: Prior to Treatment Period 1, Visit 2 (Week 1, Day 1)

a) Review selection criteria to ensure that patients are eligible to participate.

b) Ensure that patients are properly washed out of excluded medications.

c) Assign patient number to eligible patients according to randomization.

d) Perform brief physical examination of patient. Check vital signs. Record results.

e) Obtain baseline sexual experience.

3. Treatment Period 1: Visit 2 (Weeks 1-3, Days 1-21)

a) Provide patients with one bottle containing three weeks' supply of study medication according to randomization. Provide patients with one stopwatch.

b) Instruct patients and partners to attempt sexual intercourse at least once a week. Provide patients with diaries containing questionnaires and other records. Instruct patients and partners to complete a set of questionnaires at the end of the three-week period.

c) Instruct patients to record all concomitant medications and adverse events (AE) during this three-week period.

d) Remind patients to return containers and/or packaging, any unused study medication, and to bring the diaries on the next visit.

e) Schedule an appointment for patients to return for Visit 3.

f) Instruct patients on how to use stopwatch. Provide patients with an instruction sheet. Also instruct patients on completion of the log to record times.

4. Treatment Period 2: Visit 3 (Weeks 4-6, Days 22-42)

a) Collect all containers and/or packaging, any unused study medication, and all diaries.

b) Review adverse experiences and concomitant medications during Treatment Period 1. Ensure all documents are complete and legible, including the questionnaires.

c) Perform brief physical exam of patients. Check vital signs. Record results.

d) Provide patients with one bottle containing three weeks' supply of study medication according to randomization.

e) Instruct patients and partners to attempt sexual intercourse at least once a week. Provide patients with diaries containing questionnaires and other records. Instruct patients and partners to complete a set of questionnaires at the end of the 3-week period.

f) Remind patients to return containers and/or packaging, unused study medication, and to bring the diaries on the next visit.

g) Schedule appointment for patients to return for Visit 4.

5. Final/Exit Visit: Visit 4 (Week 7, Days 43-49)

a) Conduct physical examination.

b) Obtain 14 ml blood from the patients for CBC and blood chemistries. Record results.

c) Collect all containers and/or packaging, any unused study medication, and all diaries. Review diaries for concomitant medication use and adverse experiences. Ensure all documents, including the questionnaires, are complete and legible.

d) Discharge patients and partners from the study.

The study summary data and time to ejaculate date are presented in Tables 9 and 10 respectively.

TABLE 9
Study Summary of 6, 9, and 12 mg Paroxetine Capsules

| | | | |
|---|---|---|---|
| | | | Reason for |
| | | | Early |
| Clinical | Patients | Patients | Termination |

| Site | Enrolled | Completed | AE* | Other |
|---|---|---|---|---|
| A | 10 | 10 | 0 | 0 |
| B | 16 | 9 | 3 | 4 |
| C | 34 | 27 | 1 | 6 |
| D | 6 | 3 | 1 | 2 |
| E | 22 | 17 | 1 | 4 |
| F | 19 | 12 | 2 | 5 |
| TOTALS | 107 | 78 | 8 | 21 |

*AE = Adverse Experience

TABLE 10
Time to Ejaculate after 6 Weeks Treatment
Preliminary data for Baseline Latency ≦ 2 Minutes

| | | | Paroxetine Dosage/Capsule | | |
|---|---|---|---|---|---|
| | Reported By | Baseline* | 6 mg | 9 mg | 12 mg |
| Mean Time | Patient | 1 | 2.4 | 2.4 | 2.4 |
| (minutes) | Partner | 1.2 | 2.6 | 2.3 | 3.5 |
| Range | Patient | 0.03-2 | 0.03-16 | 0.03-12.5 | 0.25-10 |
| (minutes) | Partner | 0.03-10 | 0.4-15 | 0.03-10 | 0.12-30 |
| n | Patient | 87 | 25 | 25 | 24 |
| | Partner | 84 | 23 | 23 | 23 |

*Combined Data

The foregoing is intended to be illustrative of the present invention, but not limiting. Numerous variations and modifications of the present invention may be effected without departing from the true spirit and scope of the invention.

We claim:

1. A kit suitable for treating premature ejaculation dysfunction in a human male patient which comprises a package of discrete dosage forms of paroxetine or pharmaceutically acceptable salt thereof, the package comprising an articulated strip of calendar cards arranged for a designated oral therapy regimen of dosages comprising in sequence:
a paroxetine starter dosage strip containing seven paroxetine-containing dosage forms arranged in spaced apart relationship to one another on said strip to be taken daily orally on days 1-7 of an initial loading period and dosage day indicia for correlating a particular paroxetine starter dosage to a particular day on a one to one starter dosage form relationship;
a paroxetine optimization dosage strip containing at least 14 and up to and including 28 paroxetine-containing dosage forms arranged in spaced apart relationship to one another on said strip to be taken daily orally on day 8 through the last day of an optimization period of about 14 to about 28 days, and dosage day indicia for correlating a particular paroxetine optimization dosage to a particular day on a one to one optimization dosage form relationship, the amount of the paroxetine optimization dosage per optimization dosage form being twice that of the paroxetine starter dosage per starter dosage form; and
a paroxetine continual maintenance dosage strip containing a plurality of paroxetine-containing dosage forms arranged in spaced apart relationship to one another on said strip, the amount of paroxetine dosage in at least every other continual maintenance dosage form being the same as the paroxetine starter dosage, each continual maintenance dosage form to be taken orally ad lib within a period of about two to about 20 hours prior to engaging in sexual intercourse.

2. The package of claim 1 wherein the amount of said starter dosage form and at least every other said continual maintenance dosage form contains about 5 to about 40 milligrams paroxetine.

3. The package of claim 1 wherein the amount of said starter dosage form and at least every other said continual maintenance dosage form contains about 10 to about 30 milligrams paroxetine.

4. The package of claim 1 wherein each optimization dosage form contains about 10 to about 40 milligrams paroxetine.

5. The package of claim 1 containing sufficient optimization dosage forms for days 8-21.

6. The package of claim 1 containing sufficient continual maintenance dosage forms for at least one month.

7. The kit of claim 1 containing at least a three month supply of continual maintenance dosage forms.

8. The kit of claim 1 wherein the dosage forms are tablets.

9. The kit of claim 1 wherein the dosage forms are capsules.

10. The kit of claim 1 including informational literature explaining the oral therapy regimen.

11. A method for treating premature ejaculation dysfunction in a human male patient comprising accessing the kit of claim 1 and administering said designated oral therapy regimen of dosages to said patient.

12. A method for treating premature ejaculation dysfunction in a human male patient which comprises administering paroxetine or pharmaceutically acceptable salt thereof to said patient according to the following oral therapy regimen:
(A) first administering daily a starter dosage of a single dosage form containing paroxetine over an initial loading time period of about seven days; then
(B) administering daily an optimization dosage of a single dosage form containing twice the amount of paroxetine as the starter dosage over an optimization time period of about two to about four weeks; and then
(C) administering a continual maintenance dosage of a single dosage form containing the same amount of paroxetine as the starter dosage within a period of about two hours to about 20 hours prior to engaging in sexual intercourse.

13. The method of claim 12 wherein paroxetine in step (C) is administered at least every other day.

14. The method of claim 12 wherein paroxetine is administered as paroxetine hydrochloride.

15. The method of claim 12 wherein the optimization period is about three weeks.

16. The method of claim 12 where in step (C) said paroxitine administration is within a period of about three to about six hours.

17. The method in accordance with claim 12 wherein each of the starter and continual maintenance dosage amount is about 5 to about 40 milligrams.

18. The method in accordance with claim 12 wherein each of the starter and continual maintenance dosage amount is about 10 to about 30 milligrams.

19. The method of claim 12 wherein the optimization dosage amount is about 10 to about 40 milligrams and each of the starter and maintenance dosage amount is half the optimization dose.

20. A method suitable for treating premature ejaculation dysfunction in a human male patient which comprises orally administering to said patient, at least every other day, within about two to about 20 hours prior to sexual intercourse, paroxetine or a pharmaceutically acceptable acid addition salt thereof in a continual maintenance dosage amount sufficient to maintain a delay in the onset of ejaculation from pre-treatment ejaculation time by the patient during subsequent sexual intercourse.

21. The method in accordance with claim 20 wherein the dosage amount is in the range of about 5 to about 40 milligrams.

22. The method in accordance with claim 20 wherein the dosage amount is in the range of about 10 to about 30 milligrams.

23. The method in accordance with claim 20 further comprising the step of orally administering to the patient a daily starter dosage of about 5 to about 40 milligrams paroxetine over an initial loading period of a duration sufficient to delay onset of ejaculation prior to the patient converting to continual maintenance dose administration.

24. The method in accordance with claim 23 wherein said starter and continual maintenance dosage is the same amount.

25. The method in accordance with claim 20 wherein paroxetine is administered as paroxetine hydrochloride.

26. The method of claim 20 wherein administration is within a period of about three to about six hours.

27. The kit of claim 1 wherein each of the dosage forms contains paroxetine hydrochloride.

28. The kit of claim 1 wherein at least every other one of the continual maintenance dosage forms contains paroxetine hydrochloride.
